(19) United States
(12) Patent Application Publication
(10) Pub. No.: US 2005/0101621 A1
(43) Pub. Date: May 12, 2005

(54) Method for rapid detoxification of addiction

(76) Inventor: Richard Lipsky, Westwood, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/706,765

(22) Filed: Nov. 12, 2003

(51) Int. Cl. A61K 31/485

(52) U.S. Cl. 514/282

(57) ABSTRACT

A process for general anesthesia assisted opiate detoxification comprises administering an opiod antagonist at at least a rate of 0.6 mg/kg/hr to an addicted patient.

### BACKGROUND OF THE INVENTION

The present invention is related to detoxifying subjects that are addicted to opiods or opiates.

Opiates are generally thought of as a group of narcotic analgesics derived from opium. Opiods, also known as opiod agonists, are a group of drugs that exhibit opium or morphine-like properties and that are used primarily as moderate to strong analgesics. Both opiates and opiods, whether used in a clinical or non-clinical environment, are highly addictive and, in addition to acting as an analgesic, produce other effects such as sedation, slowed reflexes, sluggish movement, respiratory depression, mood changes, and mental clouding, usually without the loss of consciousness.

Throughout the remainder of this disclosure the term “opiod” will be used to refer to any narcotic analgesic that produce these effects and includes, but is not limited to the following: opium, codeine, morphine, heroin, hydromorphone, oxycodone, methadone, oxymorphone, hydrocodone Darvon, Talwin, etc.

Opiods act as agonists, interacting with stereospecific and saturable binding sites in the brain and other tissues. In fact, the term “opiods” was coined to generally refer to all exogenous substances that bind stereospecifically to any of several subspecies of opiod receptors and produce agonist actions.

Addiction is a behavioral pattern of drug use, characterized by a compulsive use of a drug, the securing of its supply, and a high tendency to relapse after withdrawal. The abuse potential of an opiod is established by at least a few factors including the ability of the drug to produce the kind of physical and psychological dependence that makes withdrawal unappealing enough to cause drug seeking activity, the length of time required for withdrawal and the severity of withdrawal symptoms.

Treatment of addiction seeks to remove the addicting drug from the subject's body and prevent the subject from reestablishing a dependence on the drug. Treatment, however, has proven to be a difficult medical and social problem because addicts have a great likelihood of remaining addicted or relapsing. The success of treating an addict usually involves determining whether treatment is appropriate for a given subject or patient, minimizing the length of time required for treatment, controlling the symptoms associated with withdrawal, and monitoring and counseling to prevent relapse. The length of time required for treatment and controlling withdrawal symptoms have been found to be addressable through the administration of certain pharmacological agents.

In particular, research using analogs of opium revealed that chemical changes in the structure of opium resulted in agents that produce agonistic, antagonistic, or a combination of agonistic and antagonistic effects in a subject. The different effects was determined to be due to preferential binding of the analogs to different opiate receptors. Additional research has shown that the analogs compete with the addictive opiods for the receptors. Thus, the time for treatment may be reduced by increasing the rate at which the competing agents occupy the opiate receptors, thereby leaving less receptors for the addictive agent. Consequently, more of the addictive agent will be in unbound form and excreted from the subject's body.

It has been found that withdrawal symptoms are inverse in proportion to the rate at which the addictive agent is displaced at the receptor sites. That is, the faster the displacement rate the more severe are the withdrawal symptoms. On the other hand, the faster displacement rate the faster the patient becomes drug free resulting in a short period for the existence of withdrawal symptoms.

One treatment involving the administration of pharmacological agents involves the use of a therapeutic agent that completely binds to the same receptors as the addictive agent. In this approach, the therapeutic agent acts as an antagonist with the addictive target agent been the agonist. The antagonist agent displaces the agonist agent, but has little addictive potential of its own, because it does not activate the receptors. This approach has the promising effect of reducing the concentration of the target drug in the subject's body at a very rapid rate and therefore greatly reduces the time for the subject to be drug free. However, the very rapid removal rate of the addictive agent results in exaggerated withdrawal symptoms. Opiate antagonists that are commonly used in this type treatment include Naloxone, Naltrexone, and others.

To combat the exaggerated withdrawal symptoms associated with the use of these antagonists, treatment usually includes anesthetizing the patient and limiting the dosage to very low levels over a relatively long period. Increasing the dosage of an opiate antagonist results in a reduction of the treatment time. However, it is generally thought that administering Naloxone at a rate greater than 1.5 mg/hour or giving more than 4 mg in a single dosage exposes the patient to severe medical risks, including loss of life. For that reason, and others, great care is taken to the limit the rate and dosage of Naloxone in treating addicts by this method.

### SUMMARY OF THE INVENTION

The present invention is a method for opiate detoxification comprising administering an opiod antagonist at a rate greater than 0.4 mg/kg/hr. In accordance with the method, an opiod antagonist is administered at a relatively high rate, which results in the active treatment period lasting approximately one hour. The method allows for ultra-rapid detoxification of subjects addicted to opiods. In the preferred embodiment the opiod antagonist is administered for one hour and the patient spends approximately 24 hours in the care of the attending physician or treatment facility.

In the preferred embodiment the opiod antagonist is preferably administered at a rate between approximately 0.6 mg/kg/hr and 1.4 mg/kg/hr.

Further in accordance with the invention, the opiod antagonist is administered at a rate greater than 4 mg/hr.

In accordance with a preferred embodiment the opiod antagonist comprises Naxloxone. In accordance with another embodiment the opiod antagonist comprises Revex. However, any other opiod antagonist may be used in accordance with the present invention.

In addition, simultaneous with the administration of the opiod antagonist, Precedex is administered at a rate greater than 1.5 mg/kg/hr.

In accordance with another aspect of the present invention a process for detoxifying a patient addicted to an opiate comprises anesthetizing the patient and administering an opiod antagonist at a rate of at least 0.6 mg/kg/hr.

Further in accordance with this aspect of the invention at the end of infusion or administration of the opiod antagonist the patient is given Revex, most preferably 8 mg of Revex.

The process may further include giving valium, xanax and trazedone orally to the patient prior to anesthetizing the patient.

In accordance with yet another aspect of the present invention, a process for opiate dependency detoxification comprises anesthetizing and intubating a patient. The cardiovascular activity of the patient is then stabilized and an opiod antagonist is administered to the patient at a rate greater than 0.6 mg/kg/hr.

In accordance with this aspect of the invention the patient is given Valium, Xanax and Trazedone orally prior to anesthetization.

Further in accordance with this aspect, stabilizing the cardiovascular activity of the patient comprises administering and titrating Precedex to the patient.

### DETAILED DESCRIPTION

The present invention is a process or method for opiate detoxification. As is discussed in greater detail below, in a preferred embodiment the invention is method for general anesthesia assisted opiate detoxification. In accordance with another embodiment, the method comprises administration of an opiod antagonist in a single dosage without the administration of general anesthesia. In accordance with the present invention an opiate includes the following addictive agents: opium, codeine, morphine, heroin, hydromorphone (Dilauid), oxycodone (Percodan), oxymorphone (Numorphan), meperidine (Demerol), methadone (Dolophine), hydrocodone (Vicodin), Darvon, Talwin and any other analog derived from opium.

The process begins with pretreatment to determine if treatment is appropriate for a given patient. Pretreatment also includes taking an addicted patient off the addictive agent or opiate.

After pretreatment, treatment begins with the patient being anesthetized. Treatment continues with the administration of an opiate antagonist, e.g., Naloxone, at a rate of 0.6 milligram per kilogram of the patient's weight per hour (mg/kg/hr).

After treatment, the patient enters post-treatment which essentially consists of administering mild doses of pain killers, e.g., Revex, Xanax and Vioxx and monitoring the patient.

Pretreatment is usually instantiated when a patient contacts a physician, psychiatrist or psychologist for opiate addiction treatment. Typically, a psychologist or psychiatrist makes a determination of whether the patient is a suitable candidate for opiate detoxification and rehabilitation. If the patient is determined to be a candidate, the patient is then seen by an anesthesiologist to evaluate if the patient is an appropriate candidate for general anesthesia assisted detoxification. Such an evaluation includes performance of certain pre-admission tests including CBC, blood chemistry, liver function tests, ECG, CX and X-rays. These and other pre-admission tests are conducted as is known in the art.

If the results of the pre-admission tests indicate that the patient is a candidate, the patient is then scheduled for treatment. The patient is required to stop using the addictive agent, e.g., “street drugs,” for at least twenty fours prior to the scheduled treatment date and placed on any interim substitute opiate medication. Such opiate medication may include morphine, oxypontin or any other such opiate medication. In addition, approximately twenty-four hours prior to the scheduled treatment date the patient fasts, being allowed to drink only water. At a minimum the patient is allowed to drink only water from 12:00 noon of the day preceding the day scheduled for admission. On the day scheduled for treatment the patient is examined by an internist to gain final clearance for treatment.

Treatment begins by providing the patient with Valium, Xanax and Trazedone, which are taken orally. The patient's vitals are then monitored using such equipment as an electrocardiogram, pulse oxymeter, blood pressure monitor, ETCO₂ monitor, oxygen sensor, and tidol volume monitor. An intravenous (IV) line is then set-up and general anesthesia is induced, preferably using Valium, Propofol and Mivacron. These anesthetizing agents are suited to facilitate endotracheal (ET) intubation.

Once the anesthesia takes effect, an endotracheal tube is appropriately placed through the patient's oral cavity and the patient retrieves to spontaneous respirations over the next few minutes. A Precedex infusion is then started, preferably at 1.5 mg/kg/hr, to achieve cardiovascular stability. Next antiemetics and antiperistaltic agents are then administered. In accordance with the preferred method the antiemetics and antiperistaltic agents comprise dexamethazone somatostatin and robinul; other similar agents can be employed. An antihistamine agent, e.g., Benedryl, is then given.

Next, a Naloxone (or its generic counterpart Narcane) infusion is started at rate of 0.6 mg/kg/hr for one hour, i.e., 0.6 mg/hr per unit of the patient's metric weight. In accordance with the present invention, Naloxone is administered at rate greater than 1.5 mg/hr. For example, for a female weighing approximately 50 kg (110 pounds), the rate of Naloxone infusion is 30 mg/hr. For a male weighing approximately 75 kg (165 lbs), the rate of Naloxone infusion is approximately 50 mg/hr. I have found that administration of Naloxone in accordance with the process set forth herein does not expose a patient to a severe medical risk even at these high dosages. In fact, I have found that Naloxone may be administered at a rate of 1.4 mg/kg/hr without exposing the patient to unnecessary risks. Furthermore, in an alternate embodiment a patient may be administered Naloxone in a single dosation of 50 mg, e.g., a single 50 mg injection for a 70 kg patient.

Although in the preferred method Naloxone is the opiod antagonist of choice, it is also possible to use other opiod antagonists, including Revex (nalmefene hydrochloride). In accordance with another embodiment of the invention, the method includes administering Revex at approximately 0.32 mg/kg/hr; administration of Revex at rates greater than 0.32 mg/kg/hr is also possible. Revex is currently considerably more expensive than Naloxone and for this reason Naloxone is currently the antagonist of choice. However, Revex may be used just as effectively. Furthermore, as other opiod antagonists are developed they may also be used in accordance with the methods discussed herein.

At the end of infusion of the Naloxone, 8 mg of Revex is administered intravenously. Throughout the treatment process general anesthesia is maintained with the infusion of Propofal, Precedex and intermittent boluses of Valium. After approximately one hour the infusion of the opiod antagonist, e.g., Naloxone, is stopped. The Precedex infusion is then tapered off and the patient's stomach and ET tube are suctioned to remove any excess secretion. Thereafter the patient is extubated and the treatment process ends.

The patient is then transferred out of the treatment area and Valium continues to be administered intravenously as needed. The patient is then kept overnight and monitored. To assure proper staff attention, it is generally recommended that nurse to patient ratio is no more than 1:3. The patient is usually provided with a sleeping agent, such as Ambien. On the next morning after treatment, the patient is examined by the attending physician and another dose of Revex is administered intravenously. The patient may also be given, by mouth, Xanax and Vioxx for muscle pains. The patient is then discharged and monitored by the attending physician for one week, e.g., daily phone calls, to attend to any medical problems. For approximately six months thereafter the patient is monitored by a psychologist or psychiatrist.

I have found that patients treated in accordance with the present invention do not suffer the same level of affects of withdrawal as is experienced by now known methods. In addition, contrary to generally known methods administering a high amount of an antagonist agent does not expose the patient to severe side effects as previously thought.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for opiate detoxification comprising administering an opiod antagonist at a rate greater than 0.4 mg/kg/hr.

2. The method of claim 1 wherein said opiod antagonist is administered at a rate greater than 4 mg/hr.

3. The method of claim 1 wherein the opiod antagonist is administered at rate greater than 0.6 mg/kg/hr but less than or equal to 1.4 mg/kg/hr.

4. The method of claim 1 further comprising simultaneously administering Precedex at a rate greater than or equal to 1.5 mg/kg/hr.

5. The method of claim 1 wherein said opiod antagonist is administered in a single dosage of 50 mg.

6. The method of claim 1 wherein said opiod antagonist is Naloxone.

7. The method claim 6 wherein the Naloxone is administered at a rate greater than 4 mg/hr.

8. The method of claim 6 wherein the Naloxone is administered at rate greater than 0.6 mg/kg/hr but less than or equal to 1.4 mg/kg/hr.

9. The method of claim 6 further comprising simultaneously administering Precedex at a rate greater than or equal to 1.5 mg/kg/hr.

10. The method of claim 6 wherein the Naloxone is administered in a single dosage of 50 mg.

11. The method of claim 1 wherein said opiod antagonist is Revex.

12. A method for opiate detoxification comprising administering Revex at a rate greater than or equal to 0.32 mg/kg/hr.

13. A method for detoxifying a patient addicted to an opiate, comprising:
anesthetizing the patient; and
administering an opiod antagonist selected from the group consisting of Naloxone and Revex at a rate of at least 0.6 mg/kg/hr.

14. The method of claim 13 further comprising simultaneously administering Precedex at a rate of at least 1.5 mg/kg/hr.

15. The method of claim 13 further comprising stopping said administering of said opiod antagonist and giving the patient Revex.

16. The method of claim 15 further comprising giving 8 mg of Revex to the patient.

17. The method of claim 13 further comprising intubating the patient prior to said administering of an opiod antagonist.

18. The method of claim 13 further comprising giving Valium, Xanax and Trazedone orally prior to anesthetizing the patient.

19. The method of claim 13 further comprising giving he patient antiemetics and antiperistaltic agents prior to administering an opiod antagonist.

20. The method of claim 13 further comprising giving the patient an antihistamine agent prior to administering said opiod antagonist.

21. The method of claim 13 further comprising administering an opiod antagonist for substantially one hour.

22. A process for opiate dependency detoxification, comprising:
anesthetizing a patient;
intubating the patient;
stabilizing the cardiovascular activity of the patient; and
administering an opiod antagonist selected from the group consisting of Naloxone and Revex to the patient at a rate greater than 0.6 mg/kg/hr.

23. The method of claim 22 further comprising giving Valium, Xanax and Trazedone orally prior to anesthetizing the patient.

24. The method of claim 22 further comprising giving he patient antiemetics and antiperistaltic agents prior to administering an opiod antagonist.

25. The method of claim 22 wherein anesthetizing a patient comprises giving the patient Valium, Xanax and Trazedone by mouth.

26. The method of claim 22 wherein stabilizing the cardiovascular activity of the patient comprises administering and titrating Precedex to the patient.

27. The method of claim 22 further comprising giving 8 mg of Revex to the patient.

28. The method of claim 22 further comprising giving the patient an antihistamine agent prior to administering said opiod antagonist.
